(19) United States
(12) Patent Application Publication
(10) Pub. No.: US 2021/0204837 A1
(43) Pub. Date: Jul. 8, 2021

(54) SENSOR SURFACE

(71) Applicant: VISSEIRO GMBH, Berlin (DE)

(72) Inventors: Janek Jurasch, Berlin (DE); Pirmin Kelbel, Berlin (DE)

(73) Assignee: VISSEIRO GMBH, Berlin (DE)

(21) Appl. No.: 17/273,137

(22) PCT Filed: Nov. 11, 2019
(86) PCT No.: PCT/EP2019/080908

§ 371 Date: Mar. 3, 2021

(30) Foreign Application Priority Data
Nov. 9, 2018 (LU) LU 100993

(51) Int. Cl. A61B 5/11

(52) U.S. Cl. CPC A61B 5/1102; A61B 2562/0247

(57) ABSTRACT

The invention relates to a device for detecting mechanical vibrations of human or animal tissue (111), comprising at least one sensor (1) and a viscoelastic medium (100), wherein the sensor (101) is embedded in the viscoelastic medium (100). According to the invention, the sensor and the viscoelastic medium form a sensor system, wherein the viscoelastic medium forms a surface for contact with the human or animal tissue (111), and wherein the acoustic impedance of the viscoelastic medium (100) is adjusted to human or animal tissue. The invention also relates to a method for producing this device.

### FIELD OF THE INVENTION

The invention relates to a device for detecting mechanical oscillations of human or animal tissues comprising at least one sensor and a viscoelastic medium, wherein the sensor is embedded in the viscoelastic medium. The invention further relates to a method of producing this device. The device can be used in the medical field and in the personal health care sector. Areas of application are to be found in the medical technology and lifestyle field.

### BACKGROUND TO THE INVENTION

In ballistocardiography, all movements (and mechanical oscillations) in human tissue are detected. The raw signal obtained contains information about the heartbeat, the respiration and all other mechanical movements which the person makes. In order that individual statements concerning the respective parameters are possible, the signal components have to be separated. By means of the different frequency ranges of the signal components, the signal can be electrically separated using high- and low-pass filters and the parameters can be individually detected. This signal processing takes place using hardware and software.

Numerous measuring devices and products for measuring parameters on the basis of ballistocardiography (BCG) are already know in the prior art. The latter are based on different sensors and structures for different applications. The different sensors comprise, amongst other things: piezoactive materials, differential air or water pressure and high-frequency radar.

Published international patent application WO 2017/134681 A2 discloses a system for BCG measurements using piezosensors with the approach of connecting the sensors by means of a solid plate made of wood or similar material, in order to increase the area. The sensors are applied on the material. Explicit size ratios are stated. A drawback with this disclosure is that the sensor is arranged on the surface of the plate.

In published international patent application WO 2007/061619 A1, a piezoactive sensor is described, which as a cantilever can swing freely in the air. An additional mass is provided in order to increase the sensitivity. A drawback with this disclosure is the complex structure with a separate cantilever and the required fixed structural housing, which has to enclose the cantilever.

Published German patent application DE 10 2013 110 900 A1 describes the concept of a characteristic acoustic impedance adaptation for an ultrasound application using one or more piezo-based sensors and air coupling. The drawback with this embodiment is that the entire sensor has to be enlarged to enlarge the surface.

Published international patent application WO 2016/053398 A1 discloses a seat cushion with fibre-optic sensors for detecting vital parameters. In addition, a plurality of pressure sensors are incorporated, which measure the pressure distribution when seated. The sensor system is constituted in several layers and embedded in an elastic medium, which represents a cushion. The electronics and battery are also contained. The elastic medium can be a foam material and serve solely for seating comfort.

An essential drawback of all the systems described in the prior art consists in the fact that the area over which the oscillations can be detected depends on the area of the respective sensors. Thus, if large measurement areas are required, the sensors have to be designed with a correspondingly large area. The latter are thus more susceptible to damage and furthermore the cost for the production of the systems is increased considerably.

### Problem of the Invention

It is the problem of the present invention to make available a device of the type mentioned at the outset, which avoids the drawbacks of the solutions known from the prior art.

### SUMMARY OF THE INVENTION

The problem is solved by a device of the type mentioned at the outset, wherein the sensor and the viscoelastic medium represent a sensor system, wherein the viscoelastic medium represents a surface for contacting the human or animal tissue, and wherein the characteristic acoustic impedance of the viscoelastic medium is adapted to human or animal bodies. According to the invention: the device comprises a sensor system which is essentially formed by a viscoelastic medium (e.g. gel), which picks up the oscillations to be detected and relays the latter to at least one sensor (oscillation transducer). According to the invention, therefore, the viscoelastic medium is part of the sensor system and provides with its surface the active measurement area. The contact with human or animal tissue can be produced directly and/or indirectly, i.e. for example via a foil, a piece of clothing, other textiles or suchlike. Moreover, the oscillations (e.g. sound waves) are transmitted through the viscoelastic medium, so that the entire device or sensor system does not have to be set into oscillation. This has the advantage that less energy is required with the same signal quality and area ratio. The advantageous properties are enabled by the setting of the mechanical transmission properties of the gel, i.e. in particular by the fact that the characteristic acoustic impedance of the viscoelastic medium is adapted to human or animal bodies. According to the invention, the viscoelastic medium must therefore have a specific characteristic acoustic impedance corresponding to the application. The characteristic acoustic impedance can be set or determined, amongst other things, by the bulk modulus, the density and the shear modulus. The sensor serves within the sensor system solely as an oscillation transducer, so that the measurement area and the quality/sensitivity of the device according to the invention are not dependent on the size or area of the sensor. The point at which the sensor is embedded in the viscoelastic medium makes no difference. The surface of the sensor can be much smaller than the sensitive surface of the entire sensor system or of the viscoelastic medium. That is to say that the viscoelastic medium forms the (large) sensitive surface for the contact with human or animal tissue, whereas only a much smaller area is sufficient for the sensor. The production costs for the device according to the invention can thus be reduced considerably. In particular, large measurement areas can also be produced in a cost-effective manner.

In an exemplary embodiment of the invention, the sensor can advantageously be a pressure sensor. For example, the sensor can be made from a piezoactive material.

In a further exemplary embodiment of the invention, provision is made such that the sensor is planar and sensitive to signals at both sides. This is especially advantageous when the sensor is embedded in the viscoelastic material free-floatingly.

For example, the sensor can have a thickness of less than 1 mm and/or an area of less than 100 cm².

In a further exemplary embodiment of the invention, provision is made such that at least one side of the sensor system is designed such that a surface contact with the human or animal tissue can be established with this side. At least one side of the sensor system can thus be formed such that a large or full area contact with the human or animal tissue can be established with this side.

The sensor system can for example have a thickness of up to 100 mm.

In an advantageous embodiment of the invention, provision is further made such that the viscoelastic medium is made of a homogeneous material. The viscoelastic gel should be homogeneous, in order to achieve the oscillation transmission (e.g. acoustic transmission) described above. In particular, it must not be heterogeneous (such as for example foamed material), since heterogeneities occur at the boundary layers, which have an adverse effect on or interfere with the signal quality. “Homogeneous” in the sense of the invention thus means that the corresponding material does not contain any particles, inclusions, gas bubbles, liquid droplets, lumps, impurities or other elements interfering with the homogeneity, which could adversely affect the transmission of oscillations (e.g. sound waves).

In an optimum adaptation of the characteristic acoustic impedance of the viscoelastic medium to human or animal bodies, a value range for the characteristic acoustic impedance of the viscoelastic medium of 1400-2000 kNs/m³ results.

In a further exemplary embodiment of the invention, provision is made such that the viscoelastic medium has a density in the range from 900 to 1100 kg/m³, a hardness of 15 shore A to 40 shore A and a characteristic acoustic impedance between 1400 and 2000 kNs/m³. These properties of the viscoelastic medium, preferably a gel, are advantageously adapted to vital parameters and contacting of the human or animal tissue for example through clothing.

For example, the viscoelastic medium can be a component of the group consisting of carbamates, polyols, polyisocyanates and silicones, wherein the medium can be water-free.

In a further exemplary embodiment, provision is made such that the area ratio of sensor surface and contact surface of the sensor system is at most 1:4. According to the invention, the surface of the sensor (oscillation transducer) is therefore much smaller than the sensitive surface of the entire sensor system or the surface of the viscoelastic medium. That is to say that the viscoelastic medium forms a large surface, whereas only a relatively small area is required for the sensor. The size ratio between the sensor surface and the surface of the viscoelastic medium can amount for example to at least 4:1, preferably 5:1, particularly preferably 10:1, more preferably 25:1, in particular 50:1.

In a further exemplary embodiment of the invention, provision is also made such that the at least one sensor can comprise a transmitter unit. In a further embodiment, the sensor can be connected to an evaluation unit, for example by cable or cableless.

Furthermore, the viscoelastic medium can be surrounded by a polyurethane foil and/or textiles for protection.

In a further exemplary embodiment, provision is made such that a part of the surface of the viscoelastic medium is covered with a bordering material, wherein the characteristic acoustic impedances of the viscoelastic medium and of the bordering material are adjusted at their boundary layer according to the in equation

$$\vert \frac{{Z}_{M 3} - {Z}_{M 2}}{{Z}_{M 3} + {Z}_{M 2}} \vert > 0.85$$

with Z_{M2}=characteristic acoustic impedance of the viscoelastic medium and
Z_{M3}=characteristic acoustic impedance of the bordering material.

With this embodiment of the device according to the invention, optimum decoupling of the sensor system in respect of the surroundings or interfering influences is ensured. The decoupling is achieved via the characteristic acoustic impedance of the materials at the boundary layer between the viscoelastic medium and the bordering material. Depending on the application, the sensor system can be surrounded for example with a foam for this purpose, so that only a contact surface of the sensor system is in contact with the human or animal tissue. The foam is used for the decoupling of the measurement system with respect to disturbances from outside, since mechanical oscillations are damped almost completely by the foam. The bordering material can for example surround the sensor system or be applied on its surface in such a way that only one side or partial area remains uncovered or free for the contact with the human or animal body, whereas all the other sides or partial areas are completely covered by the bordering material.

According to the invention, the problem is also solved by a method for producing a device for detecting mechanical oscillations of human or animal tissues, wherein at least one sensor is embedded in a viscoelastic medium, wherein the characteristic acoustic impedance of the viscoelastic medium is adapted to human or animal bodies.

In a preferred embodiment of the method according to the invention, provision is made such that the sensor is embedded in the viscoelastic medium free-floatingly, so that it is completely surrounded by the viscoelastic medium at the end of the production process.

The adaptation of the characteristic acoustic impedance of the viscoelastic medium to human or animal bodies takes place within a value range for the characteristic acoustic impedance of the viscoelastic medium of 1400-2000 kNs/m³. In particular, the viscoelastic medium can be selected and/or set in such a way that it has a density in the range from 900 to 1100 kg/m³, a hardness from 15 shore A to 40 shore A and a characteristic acoustic impedance between 1400 to 2000 kNs/m³.

The size ratio between the sensor surface and the surface of the viscoelastic medium can be designed for example such that it amounts to at least 4:1, preferably 5:1, particularly preferably 10:1, more preferably 25:1, in particular 50:1. The surface of the sensor (oscillation transducer) can thus advantageously be kept much smaller than the sensitive surface of the entire sensor system or the surface of the viscoelastic medium. The production costs for the device according to the invention can thus be considerably reduced. In particular, large measurement areas can also be created in a cost-effective manner.

In an exemplary embodiment of the production process according to the invention, provision is advantageously made such that a part of the surface of the viscoelastic medium is covered with a bordering material, wherein the characteristic acoustic impedances of the viscoelastic medium and the bordering material at their boundary layer are set corresponding to the in equation

$$\vert \frac{{Z}_{M 3} - {Z}_{M 2}}{{Z}_{M 3} + {Z}_{M 2}} \vert > 0.85$$

with Z_{M2}=characteristic acoustic impedance of the viscoelastic medium and
Z_{M3}=characteristic acoustic impedance of the bordering material.

With this embodiment of the method according to the invention, optimum decoupling of the device in respect of the surroundings or disturbing influences from the surroundings is achieved. For example, the sensor system can be partially encased with a foam for this purpose, in such a way that only a contact surface of the sensor system can be in contact with the measurement object (human or animal tissue).

A further subject-matter of the present invention is a seating or reclining surface or equipment or a piece of furniture with a seating or reclining surface, which comprises a device as described above.

The device according to the invention as described above can advantageously be used to detect mechanical oscillations of human or animal bodies, in particular in the medical field and in the personal health care sector as well as in the medical technology and lifestyle field.

Further aspects, features and advantages of the present invention readily emerge from the following detailed description, in which preferred embodiments and implementations are simply represented. The present invention can also be implemented in other and different embodiments and their various details can be modified in different, obvious aspects, without departing from the teaching and scope of the present invention. Accordingly, the drawings and descriptions are to be regarded as exemplary and not as limiting. Additional problems and advantages of the invention are represented in part in the following description and become obvious in part from the description or can be inferred from the execution of the invention.

### SUMMARY OF THE FIGURES

The present invention is described and represented based on figures and examples of embodiment. It is clear to the competent person skilled in the art that the invention is not limited to the contents of the figures and examples of embodiment. In the figures:

FIG. 1 shows the overall system comprising the sensor system/seat cushion (100 and 101), the evaluation box (103) and in the connection cable (102)

FIGS. 2A-G show possible forms of the sensor system. These are only examples and do not represent a comprehensive list of all possible forms

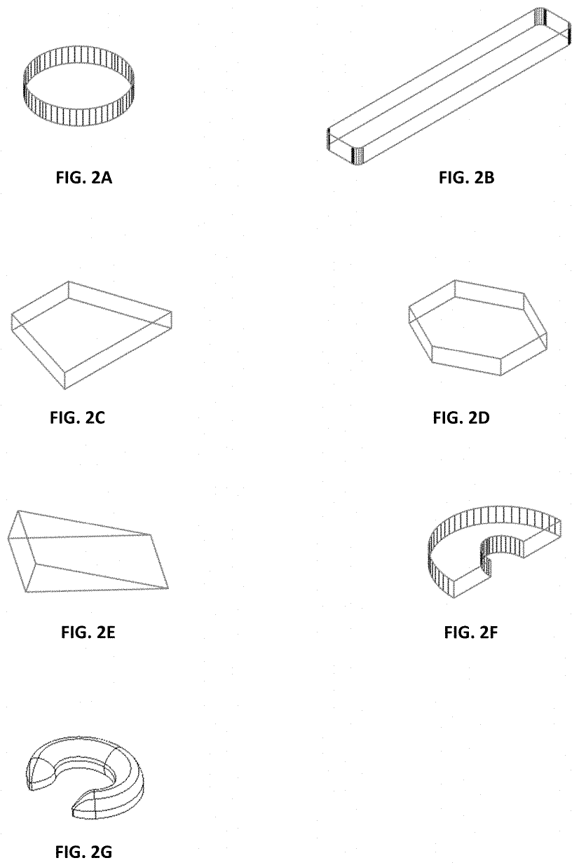

FIG. 3 shows effects of mechanical loads on the piezoactive sensor foil (101) in different embodiments

FIG. 4 shows a representation of the signal processing

FIG. 5 shows the functional principle of the characteristic acoustic impedance adaptation

### DETAILED DESCRIPTION OF THE INVENTION

The previously formulated problem of the invention is solved by the features of the independent claims. The dependent claims cover further specific embodiments of the invention.

The present invention relates to a sensor surface or the structure of a sensor system and is based on the embedding of small-area, piezoactive sensor foils (PVDF foil, piezosensors, sensor surface) into a viscoelastic medium. The sensor surface/sensor system can be used for a device, which as an independent sensor system or as an integrated module can be integrated for example into various seating and reclining furniture. The sensor system is specially adapted for measurements on living beings (man, animal). The sensor system picks up, without contact with the skin, all mechanical oscillations, in particular ballistocardiographic signals. In particular, parameters such as for example movement, respiration (breathing rate, breathing amplitude, heart rate and heart rate variability) are measured.

The embedding of the sensor foils takes place during the production process. The viscoelastic medium is cast into a mould and then completely reacts. During the reaction process, the piezoactive sensor foil can be integrated into the casting process, so that the latter is completely surrounded by the medium without air inclusions.

The novelty of the present invention lies in the structure which takes account of specific properties of the viscoelastic medium. The characteristic acoustic impedance of the medium corresponds in the ideal case to that of human tissue (see table 1). In the case of direct contact between medium and human tissue, virtually all mechanical oscillations (movements in the human tissue, acoustic oscillations) are transmitted from the human tissue into the medium.

Additional materials, for example the person's clothing or coverings for the sensor system, act as a bridge (112) during the acoustic transmission. Since the material of the bridge is not homogeneous, a characteristic acoustic impedance cannot be determined. A limitation for the bridge results from the respective application. During the application, the bridge material (for example cotton fabric) is compressed by the forces acting. When the bridge is idealised, the transmission factor or reflection factor between viscoelastic medium (100) and human (or animal) tissue (111) is ascertained as follows:

$$Reflection factor \text{:} r = \frac{{Z}_{2} - {Z}_{1}}{{Z}_{2} + {Z}_{1}} , \vert r \vert \leq 1$$

$$Transmission factor \text{:} t = \frac{2 \cdot {Z}_{2}}{{Z}_{2} + {Z}_{1}} = 1 - \vert r \vert$$

wherein Z₁ and Z₂ correspond to the characteristic acoustic impedances of two bordering materials.

TABLE 1
Characteristic acoustic impedances and density of
selected materials and structures

| | Characteristic acoustic | |
|---|---|---|
| Material | impedance Z in kNs/m³ | Density in kg/m³ |
| Water (30° C.) | 1502 | 1000 |
| Air (20° C.) | 0.4136 | 1.2 |
| Human tissue | 1573 | 940-1040 |
| (average) | | |
| Viscoelastic | 1400-2000 | 900-1100 |
| medium | | |

With the values from Table 1, the average transmission and reflection factors result as follows in Table 2:

| Boundary layer: | Reflection factor [r] | Transmission factor [t] |
|---|---|---|
| Tissue - Air | −0.9995 | 0.0005 |
| Medium - Air | −0.9996 | 0.0004 |
| Tissue - | −0.1195 | 0.8805 |
| medium | | |

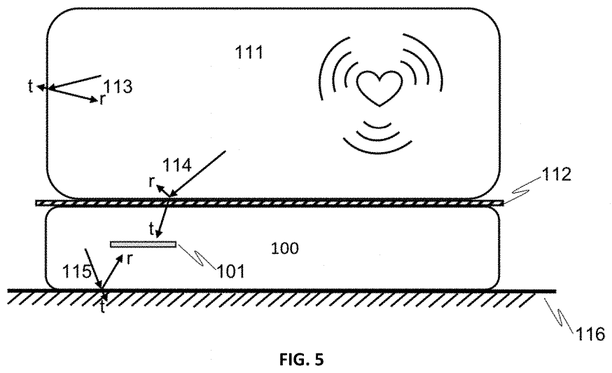

The oscillations generated by human tissue are reflected at the interface with the air and transmitted for the most part into the medium. FIG. 5 represents this principle schematically. An advantage of this invention lies in the fact that, the required piezoactive sensor surface can be considerably reduced in size by the coupling and multiple reflection of the oscillations in the medium. Thus, in an application of the sensor surface as a seat cushion, only a few square centimetres of piezoactive sensor foil are required.

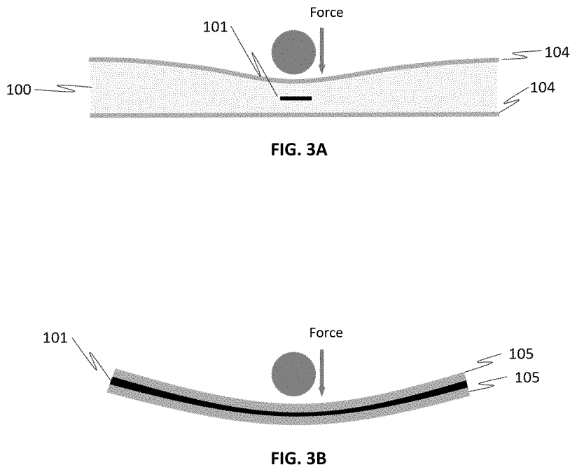

At the same time, the surface for the mechanical contact between human tissue and sensor system is established by the medium. The surface of the sensor system is flexible and deformable. The medium protects the piezoactive sensor foils against large mechanical stresses (see FIG. 3). The mechanical wear on the piezoactive sensor foils is thus reduced and the service life thus increased.

The properties to be set, in particular with regard to the physical density and the characteristic acoustic impedance of the viscoelastic medium, result from its composition during production. The production and composition of a viscoelastic medium is already known, for example the composition and the production of a viscoelastic medium based on reaction products from polyols and polyisocyanates (EP 1125975B1).

The invention is described with the aid of the represented illustrations. The figures show a measuring device according to the invention, which records all movements, mechanical oscillations and therefore also acoustic oscillations within the tissue of a living being (human or animal). It is used for the purpose of ascertaining and detecting the vitality of the living being. The derived magnitudes are at least: movement, heart rate, heart rate variability, breathing rate, breathing amplitude.

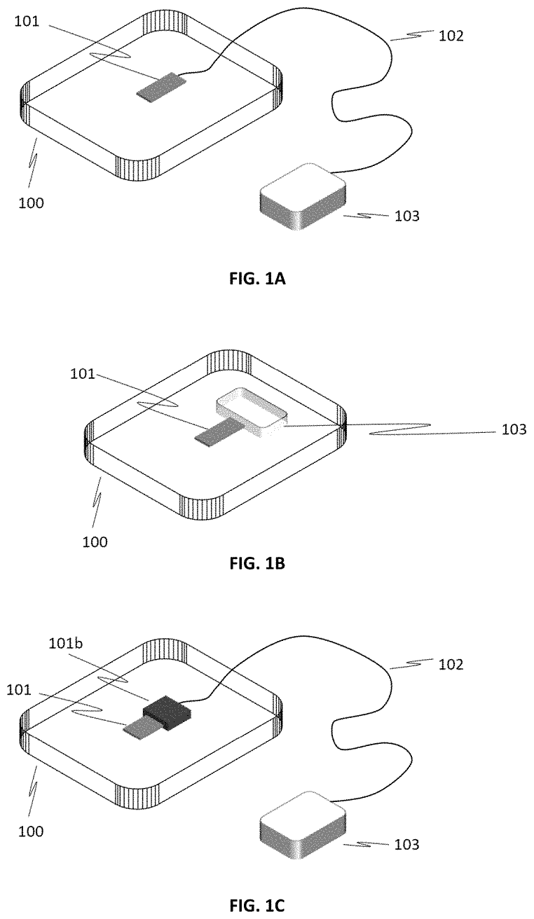

FIG. 1 shows schematically the entire structure of the sensor system. The latter comprises the sensor system (100 and 101), a connecting cable (102) and the evaluation unit (103). The power supply for the evaluation unit (103) is not represented. It can take place via an integrated power supply (battery, storage battery) or via an external power supply unit. In the case of the supply via an external power supply unit, the latter is also connected by cable to the evaluation unit. The entire evaluation electronics (103) can be wholly or partially integrated into the sensor system (100 and 101) (see FIG. 1b).

Furthermore, the structure of the sensor system (100 and 101) is represented in FIG. 1. The sensor system consists of a viscoelastic medium, which for example can be Technogel (BTGS, encased with PU foil (104, FIG. 3). Decisive properties of the medium are the mechanical properties of density and hardness.

One or more piezoactive sensor foils (101) are introduced inside the viscoelastic medium. Only one piezoactive sensor foil is represented in FIG. 1, but a plurality of piezoactive sensor foils can be introduced, which can have further advantages (signal correlation). In addition, an electrical amplifier circuit (101b) can be introduced directly on the sensor (101) or at least inside the viscoelastic medium (100) and connected to the piezoactive sensor foil. This serves to amplify the signal, or to reduce (electrical) interference noise and the susceptibility of the signal with regard to (electrical) interference noise, by means of an impedance adaptation (see FIG. 1C).

The use of the viscoelastic medium has a number of purposes: As a result of the viscous properties, locally acting forces are distributed and the stress on the piezoactive sensor foil (101) is reduced. Two schematic structures for this purpose are sketched in FIG. 3. FIG. 3A corresponds to the structure of the sensor system from FIG. 1 in a side view. The impacting force, for example a person sitting or reclining on the sensor system, primarily deforms the viscoelastic medium: The piezoactive sensor foil (101) is deformed to a lesser extent (sinking due to mechanical stress).

FIG. 3B shows the prior art for the structure of such a sensor system for ballistocardiographic measurements. The piezoactive sensor foil fills the entire thickness and width of the sensor system and is surrounded by only a thin cover layer (105) without damping properties (foil, plastic layer). An impacting force thus leads to a direct mechanical stress on the piezoactive sensor foil. In particular, mechanical deformations of the foil lead to severe ageing of the latter.

The viscoelastic medium according to the present invention is deformable. It is dimensionally stable in the presence of loading, e.g. stretchable up to an eightfold extension, and can be cast during production into any three-dimensional shape. As a result of the viscoelastic properties of the material of the present invention, the sensor system can be adapted to any shape, for example the seat shell of a chair. Piezoactive sensor foil is marginally deformed and thus mechanically stressed. Possible three-dimensional shapes of the entire sensor system are represented for example in 2A-2G. Wherein the represented shapes do not represent a comprehensive list of possible shapes.

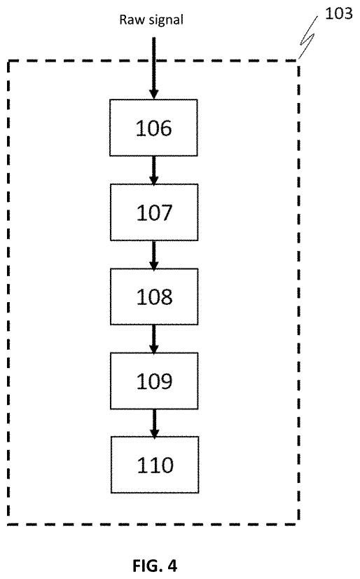

The schematic sequence of the signal processing is represented in FIG. 4. The arriving raw signal is first amplified (106). Various circuits for this purpose from the prior art are known to the person skilled in the art.

After the signal amplification, the signal is filtered with high- and low-pass filters (107). Their design and structure are also known to the person skilled in the art and have to be designed for the new sensor system. The signal is digitalised in 108 and relayed to a digital signal processor (109). Inside the digital signal processor, the signal is analysed and the parameters such as movement, heart rate, breathing rate and heart rate variability are extracted and calculated, e.g. through digital filters.

An evaluation unit with hardware and software components is required for the evaluation of the raw signals. The evaluation unit and the sensor system are a sensor system. The hardware (103) and software connected downstream of the sensor system are used for the signal processing and evaluation of the ballistocardiographic raw signals. In a possible application, the parameters are extracted and further evaluation variables are derived on the basis of the parameters obtained. In an embodiment according to the invention, these data can be sent via a wireless radio connection to a mobile terminal unit, cloud service, server, web interface, further processing ( . . . ) or displayed via a display directly on the device.

In addition, the detected individual parameters can also be combined to derive further medically relevant information. For example, a comprehensive health and fitness status. The data are then made available to the user (110). For this purpose, a display can be fitted directly to the evaluation unit or the data are transmitted by radio connection or cable-bound to further external display devices, storage media or servers and possibly stored.

FIG. 5 shows the schematic functional principle of the sensor system according to the invention. Oscillations generated by the heart (or other sources) pass through the human tissue and strike the interfaces of the human tissue. At the interface with air (113), the majority of the oscillation is reflected (“r”). At the interface (114) with the sensor system or the viscoelastic medium (100), the majority of the oscillation is transmitted and, inside the medium, strikes the piezoactive sensor foil (101), which is small in relation to the interface. At the interface (115) between the bordering material (116) and viscoelastic medium (100), the majority of the oscillation is again reflected and once again strikes the piezoactive foil (101) (at both sides). It should be noted that there must not be an impedance adaptation between the lower side of the medium (100) and the bordering material (116). The characteristic acoustic impedances of the following inquality suffice:

$$\vert \frac{{Z}_{M 3} - {Z}_{M 2}}{{Z}_{M 3} + {Z}_{M 2}} \vert > 0.85$$

Z_{M2} corresponds to the characteristic acoustic impedance of the viscoelastic medium (100)

Z_{M3} corresponds to the characteristic acoustic impedance of the bordering material (116)

Since the sensor system or the viscoelastic medium (100) is sensitive in all directions, the latter has to be decoupled from the surroundings in order to obtain a useful signal-to-noise ratio and may only have contact with the signal source (human or animal tissue (111). To carry out this decoupling, the viscoelastic medium (100) can be embedded for example in a foam bath (bordering material 116), which leaves only one side of the viscoelastic medium (100) free for the contact with the signal source. This foam bath has requirements on the characteristic acoustic impedance which are inverse with respect to the material.

An application of the invention described above arises in the personal tracking of health parameters (personnel health tracking) and the monitoring of one's own lifestyle (quantify yourself). The sensor system according to the invention can however also be used as a medical product.

Compared to the rigid structure disclosed in WO 2017/134681 A2, integration of the sensors into the surface-enlarging material is provided in the present invention, as a result of which the advantage of a multiple reflection of signals results. Furthermore, a sensor foil of the present invention is active at both sides, which results in an amplification of signals. In addition, the possibility of an impedance adaptation by the selection of the material properties is provided. A further advantage results from the fact that the material can adapt to the surface of human tissue (elastic).

With regard to the teaching of WO 2007/061619 A1, in the present invention the active sensor surface is completely surrounded by a medium adapted to a characteristic acoustic impedance. In the teaching according to WO 2007/061619, air does not offer the possibility for impedance adaptation.

With regard to the concept described in DE 10 2013 110 900 A1, according to the invention the characteristic acoustic impedance adaptation takes place between human tissue and the sensor system. A reduction of the sensor surface can thus be advantageously generated, which leads to a considerable cost reduction.

The above description of the preferred embodiment of the invention has been given for the purpose of illustration and description. It is not intended to be exhaustive or to limit the invention precisely to the disclosed form. Modifications and variations are possible in view of the above teaching and can be achieved from practising the invention. The embodiment has been selected and described to explain the principles of the invention and its practical application, in order to enable the person skilled in the art to use the invention in various embodiments which are suitable for the specific intended use. It is intended that the scope of the invention is defined by the appended claims and their equivalents. The entirety of each of the aforementioned documents is included herein by reference.

### LIST OF REFERENCES

100 Viscoelastic medium
101 Sensor
101b Amplifier circuit
102 Connecting cable
103 Evaluation unit
104 Cover layer
105 Cover layer as a direct encasement of the sensor
106 Amplification raw signal
107 Filtering signal
108 Digitalisation signal
109 Relaying of signal
110 Signal made available
111 human or animal tissue
112 Bridge
113 Interface air
114 Interface sensor system
115 Interface air/medium
116 Bordering material

1. A device for detecting mechanical oscillations of human or animal tissues, the device comprising at least one sensor and a viscoelastic medium, wherein the sensor is embedded in the viscoelastic medium, characterized in that the sensor and the viscoelastic medium represent a sensor system, wherein the viscoelastic medium represents a surface for contacting the human or animal tissue, and wherein the characteristic acoustic impedance of the viscoelastic medium is adapted to human or animal bodies.

2. The device according to claim 1, wherein the sensor is a pressure sensor.

3. The device according to claim 1, wherein the sensor is planar and sensitive to signals at both sides.

4. The device according to claim 1, wherein the sensor is embedded in the viscoelastic medium free-floatingly.

5. The device according to claim 1, wherein at least one side of the sensor system is designed such that surface contact with the human or animal tissue can be established with the side.

6. The device according to claim 1, wherein the viscoelastic medium is made of a homogeneous material.

7. The device according to claim 1, wherein the viscoelastic medium has a characteristic acoustic impedance between 1400 and 2000 kNs/m³.

8. The device according to claim 1, wherein the viscoelastic medium has a density in the range from 900 to 1100 kg/m³, a hardness of 15 shore A to 40 shore A, and a characteristic acoustic impedance between 1400 and 2000 kNs/m³.

9. The device according to claim 1, wherein the viscoelastic medium is a component of the group consisting of carbamates, polyols, polyisocyanates, and silicones.

10. The device according to claim 1, wherein the area ratio of sensor surface and contact surface of the sensor system is at most 1:4.

11. The device according to claim 1, wherein the at least one sensor comprises a transmitter unit.

12. The device according to claim 1, the viscoelastic medium being surrounded by a polyurethane foil and/or textiles for protection.

13. The device according to claim 1, characterized in that a part of the surface of the viscoelastic medium is covered by a bordering material, wherein the characteristic acoustic impedances of the viscoelastic medium and the bordering material are adjusted at their boundary layer according to the equation
$$\vert \frac{{Z}_{M 3} - {Z}_{M 2}}{{Z}_{M 3} + {Z}_{M 2}} \vert > 0.85$$
with
Z_{M2}=characteristic acoustic impedance of the viscoelastic medium (100) and
Z_{M3}=characteristic acoustic impedance of the bordering material (116).

14. A method for producing a device for detecting mechanical oscillations of human or animal tissues, with which at least one sensor is embedded in a viscoelastic medium, characterized in that the characteristic acoustic impedance of the viscoelastic medium is adapted to human or animal bodies.

15. The method according to claim 14, wherein the sensor is embedded in the viscoelastic medium free-floatingly.
